(19) United States
(12) Patent Application Publication
(10) Pub. No.: US 2004/0220135 A1
(43) Pub. Date: Nov. 4, 2004

(54) Combination therapy for treatment of neoplasia

(75) Inventor: Bruce Nathaniel Gray, Claremont (AU)

Correspondence Address: MERCHANT & GOULD PC, P.O. BOX 2903, MINNEAPOLIS, MN, 55402-0903, US

(73) Assignee: SIRTEX MEDICAL LIMITED, Burswood (AU)

(21) Appl. No.: 10/428,448

(22) Filed: Apr. 30, 2003

(51) Int. Cl. A61K 31/7072; A61K 51/00

(52) U.S. Cl. 514/50; 424/1.11

(57) ABSTRACT

The present invention concerns an unexpected synergistic combination of known antineoplastic therapies, which provides unexpectedly greater efficacy than either therapy alone in the treatment of neoplasia. Accordingly, the present invention provides a method that has utility in the treatment of various forms of cancer and tumours, but particularly in the treatment of brain and colorectal liver metastases and more specifically in the treatment of primary and secondary cancer of the liver. It is to be understood that the selective internal radiation therapies described herein should not be limited to radioactive microparticles, but may be extended to any radioactive particles that are suitable for use in the treatment methods described herein.

### FIELD OF THE INVENTION

The present invention concerns an unexpected synergistic combination of known antineoplastic therapies, which provides unexpectedly greater efficacy than either therapy alone in the treatment of neoplasia. Accordingly, the present invention provides a method that has utility in the treatment of various forms of cancer and tumours, but particularly in the treatment of brain and colorectal liver metastases and more specifically in the treatment of primary and secondary cancer of the liver. It is to be understood that the selective internal radiation therapies described herein should not be limited to radioactive microparticles, but may be extended to any radioactive particles that are suitable for use in the treatment methods described herein.

The invention further provides a synergistic combination of antineoplasia agents, comprising an effective antineoplastic amount of 5-fluorouraoil and leucovorin and an amount of radionuclide-doped microparticles suitable for selective internal radiation therapy (SIRT) to effectively treat a neoplastic growth.

The invention also provides for the use of effective amounts of 5-fluorouracil and leucovorin and an amount of radionuclide-doped microparticles suitable for SIRT to effectively treat a neoplastic growth in the preparation of a medicament for the treatment of neoplasia generally and in particular primary and secondary cancer of the liver and the brain.

### BACKGROUND ART

Neoplasia is now the second leading cause of death in the United States and is a disease characterized by an abnormal proliferation of cell growth known as a neoplasm. Neoplasms may manifest in the form of a leukaemia or a tumour, and may be benign or malignant. Malignant neoplasms, in particular, can result in a serious disease state, which may threaten life. Significant research efforts and resources have been directed toward the elucidation of antineoplastic measures, including chemotherapeutic and radiotherapeutic agents which are effective in treating patients suffering from neoplasia. Effective antineoplastic agents include those that inhibit or control the rapid proliferation of cells associated with neoplasms, those that effect regression or remission of neoplasms, and those that generally prolong the survival of patients suffering from neoplasia. Successful treatment of malignant neoplasia, or cancer, requires elimination of all malignant cells, whether they are found at the primary site, or whether they have extended to local-regional areas or have metastasized to other regions of the body.

Of the vast forms of malignant neoplasms colorectal cancer is one of the commonest. The liver is a dominant site of metastatic spread as a result of the portal venous drainage of the gut and is the main cause of death in these patients (Gilbert J. et al. (1984) Brit J Surgery. 71, 203-205). Treatment of such disease states is usually achieved with one or a combination of three therapies: surgery, chemotherapy and radiotherapy.

Surgery involves the bulk removal of diseased tissue. When tumour growth is recognized, excision of the tumour mass by surgery is regarded as the therapy of choice. So, for example, in a minority of patients with liver metastases some form of local ablation, such as surgical resection, cryotherapy or radiofrequency ablation can offer the potential for long-tern cure. However, this approach, while producing very satisfactory results as a general measure, is effective only for patients with tumours at an early stage of development. It cannot be used in, for example, the liver where the vast majority of the liver is covered with disseminated neoplastic conditions. Regardless of the developmental stage of the neoplastic mass, therapy through excision is frequently undesirable due to the possibility of missing related growths metastasized to a remote site. The physical scarring left by frequently radical surgical technique, and the risks commonly associated with surgery of any type.

Chemotherapy involves the disruption of cell replication or cell metabolism. It is used most often in the treatment of leukaemia, as well as liver, breast, lung, and testicular neoplasms. In recent years, various excellent antineoplastic compositions have been introduced into use for the chemotherapy with progressively improved results. Chemotherapeutic effects so far achieved nevertheless still remain temporary and are not always satisfactory in completely inhibiting the proliferation of neoplastic tissues and enabling patients to survive a long period of time.

The major classes of chemotherapeutic agents include alkylating agents, antimetabolites and antagonists, and a variety of miscellaneous agents (see Haskell, C. M., ed., (1995) and Dorr. R. T. and Von Hoff, D D., eds. (1994)).

The classic alkylating agents are highly reactive compounds that have the ability to substitute alkyl groups for the hydrogen atoms of certain organic compounds. The damage they cause interferes with DNA replication and RNA transcription. The classic alkylating agents include mechlorethamine, chlorambucil, melphalan, cyclophosphamide, ifosfamide, thlotepa and busulfan.

The antimetabolites are structural analogues of normal metabolites that are required for cell function and replication. They typically work by interacting with cellular enzymes. Among the many antimetabolites that have been developed and clinically tested are methotrexate, 5-fluorouracil (5-FU), floxuridine, cytarabine, 6-mercaptopurine, 6-thioguanine, deoxycoformycin, fludarabine, 2-chlorodeoxyadenosine, and hydroxyurea.

The compound 5-FU is possibly the most widely used antineoplastic drug in the world. 5-FU has been used clinically in the treatment of malignant tumours and cancer, including, for example, carcinomas, sarcomas, skin cancer, cancer of the digestive organs and liver, and breast cancer (A Comprehensive Treatise, 5, 327, Prenum Press, Cancer Res., 18, 478 (1958). Gastroenterology, 48, 430 (1965), Cancer Treat, Rep., 62, 533 (1987)). 5-FU, however, causes serious adverse reactions such as nausea, alopecia, diarrhoea, stomatitis, leukocytic thrombocytopenia, anorexia, pigmentation, and edema (Pharmacological Principles of Cancer Treatment, 195 (1982)). Further, as 5-FU is highly toxic, it is sometimes impossible to administer the compound over a prolonged period of time and therefore to achieve the desired curing affect.

Leucovorin (ie (6R,S)-5-formyl-tetrahydrofolate) has been available commercially for decades for the treatment of folic acid deficiency states (The Pharmacologic Basis of Therapeutics, 4th ed. (Goodman et al., eds.) The MacMillan Co. Toronto, pp. 1431-44 (1970)). In 1982, the first clinical reports of the usefulness of leucovorin as a modulator of 5-FU in antineoplastic treatment appeared (Machover et al., (1982) Cancer Treat Rep, 66, 1803-07). Leucovorin (LV) addition to 5-FU appeared to approximately double response rates in patients with gastrointestinal neoplasms. This result was confirmed in several subsequent studies (see Grem et at. (1987), Cancer Treat Rep. 71:1249-64). Currently, LV addition to 5-FU therapy is community standard practice in the United States.

While the combination of such drugs has vastly improved chemotherapy regimens there are still significant problems, with the use of such agents. Among the problems currently associated with the use of such agents to treat neoplastic growth are the high doses of agent required; toxicity toward normal cells, i.e., lack of selectivity; immunosuppression; second malignancies; and drug resistance.

Another side effect associated with present day therapies is the toxic effect of the chemotherapeutic on the normal host tissues that are the most rapidly dividing, such as the bone marrow, gut mucosa and cells of the lymphoid system. The agents also exert a variety of other adverse effects, including neurotoxicity; negative effects on sexuality and gonadal function; and cardiac, pulmonary, pancreatic and hepatic toxicities; vascular and hypersensitivity reactions, and dermatological reactions.

The clinical usefulness of a chemotherapeutic agent may also be severely limited by the emergence of malignant cells resistant to that drug. A number of cellular mechanisms are probably involved in drug resistance e.g., altered metabolism of the drugs, impermeability of the cell to the active compound or accelerated drug elimination from the cell, altered specificity of an inhibited enzyme, increased production of a target molecule, increased repair of cytotoxic lesions, or the bypassing of an inhibited reaction by alternative biochemical pathways. In some cases, resistance to one drug may confer resistance to other, biochemically distinct drugs. In this respect amplification of the gene encoding thymidylate synthase is related to resistance to treatment with 5-fluoropyrimidines.

In summary, chemotherapy has not made a dramatic impact on the treatment of neoplastic growths. Certain drugs and biologicals have shown considerable activity in various studies, but their effects are negated by numerous problems and disadvantages.

Radiotherapy has been used as an alternative to chemotherapy and usually relies on treatment through external beam technologies or more recently through locally administering radioactive materials to patients with cancer as a form of therapy.

In some of these, the radioactive materials have been incorporated into small particles, seeds, wires and similar related configurations that can be directly implanted into the cancer. When radioactive particles are administered into the blood supply of the target organ, the technique has become known as Selective Internal Radiation Therapy (SIRT). Generally, the main form of application of SIRT has been its use to treat cancers in the liver.

There are many potential advantages of SIRT over conventional, external beam radiotherapy. Firstly, the radiation is delivered preferentially to the cancer within the target organ. Secondly, the radiation is slowly and continually delivered as the radionuclide decays. Thirdly, by manipulating the arterial blood supply with vasoactive substances, it is possible to enhance the percentage of radioactive particles that go to the cancerous part of the organ, as opposed to the healthy normal issues. This has the effect of preferentially increasing the radiation dose to the cancer while maintaining the radiation dose to the normal tissues at a lower level (Burton, M. A. et al. (1988) Europ. J. Cancer Clin. Oncol. 24(8), 1373-1376).

When microparticles or other small particles are administered into the arterial blood supply of a target organ, it is desirable to have them of a size, shape and density that results in the optimal homogeneous distribution within the target organ. If the microparticles or small particles do not distribute evenly, and as a function of the absolute arterial blood flow, then they may accumulate in excessive numbers in some areas and cause focal areas of excessive radiation. It has been shown that microparticles of approximately 25-50 micron in diameter have the best distribution characteristics when administered into the arterial circulation of the liver (Meade, V. et al. (1987) Europ. J. Cancer & Clin. Oncol. 23, 2341).

However, if the particles are too dense or heavy, then they will not distribute evenly in the target organ and will accumulate in excessive concentrations in areas that do not contain the neoplastic growth. It has been shown that solid, heavy microparticles distribute poorly within the parenchyma of the liver when injected into the arterial supply of the liver. This, in turn, decreases the effective radiation reaching the neoplastic growth in the target organ, which decreases the ability of the radioactive microparticles to kill the tumour cells. In contrast, lighter microparticles with a specific gravity of the order of 2.0 distribute well within the liver (Burton, M. A. et al. (1989) Europ. J. Cancer Clin. Oncol 25, 1487-1491).

For radioactive particulate material to be used successfully for the treatment of neoplastic growth, the radiation emitted should be of high energy and short range. This ensures that the energy emitted will be deposited into the tissues immediately around the particulate material and not into tissues that are not the target of the radiation treatment. In this treatment mode, it is desirable to have high energy but short penetration beta-radiation, which will confine the radiation effects to the immediate vicinity of the particulate material. There are many radionuclides that can be incorporated into microparticles that can be used for SIRT. Of particular suitability for use in this form of treatment is the unstable isotope of yttrium (Y-90). Yttrium-90 decays with a half-life of 64 hours, while emitting a high energy pure beta radiation. However, other radionuclides may also be used in place of Y-90 of which the isotopes of holmium, samarium, iodine, iridium, phosphorus, rhenium are some examples.

The technique of SIRT has been previously reported (see, for example, Chamberlain M, et al (1983) Brit. J. Surg., 70: 596-598; Burton MA, et al (1989) Europ. J. Cancer Clin. Oncol., 25, 1487-1491; Fox RA, et al (1991) Int J. Red. Oncol. Biol. Phys. 21, 463-467; Ho S et al (1996) Europ J Nuclear Med. 23, 947-952; Yorke E, et al (1999) Clinical Cancer Res, 5 (Suppl). 3024-3030; Gray B N, et al. (1990) Int. J. Rad. Oncol. Biol. Phys, 18, 619-623). Treatment with SIRT has been shown to result in high response rates for patients with neoplastic growth in particular with colorectal liver metastases (Gray B. N. et al (1989) Surg. Oncol, 42, 192-196; Gray B, et al. (1992) Aust NZ J Surgery, 62, 105-110; Gray B N et al. (2000) Gl Cancer, 3(4), 249-257: Stubbs R, et al (1998) Hepato-gastroenterology Suppl II, LXXVII). Other studies have shown that SIRT therapy can also be effective in causing regression and prolonged survival for patients with primary hepatocellular cancer (Lau W, et al (1994) Brit J Cancer 70, 994-999; Lau W, et al. (1988) Int J Rad Oncol Biol Phys. 40, 583-592). Although SIRT is effective in controlling the liver disease, it has no effect on extra-hepatic disease.

Recently, clinicians have fried to improve the response of cancer patients by combining two or more anti-tumour therapies into a single therapeutic regimen. By combining two or more therapies, most often with different mechanisms of action, the clinician is able to both increase the therapeutic index of the individual treatments, and at the same time reduce the toxic effects to the patient.

One example of such combination therapy are the randomised clinical trials carried our by Gray et al where they compare treatment of floxuridine either with or without the addition of a single dose of radioactive microparticles (Gray et al (2001) Annals of Oncology 12: 1711-1720). Results from these studies have shown that the addition of radioactive microparticles increased the response rate from 17.6% to 44% and the time to disease progression from 9.7 months to 15.9 months. An important finding from this trial was that although most patients eventually succumbed to their disease, the liver metastases were not the primary cause of death for most patients treated with SIRT.

Combination therapies now being tested use drugs with dissimilar mechanisms of action, based on the rationale that targeting two independent pathways will result in enhanced cytotoxicity, whether additive or synergistic. The results of these experiments are entirely unpredictable as the use of two entirely different therapies usually means that each therapy works independent of the other and thus would not be expected to interact to improve the other.

Combination treatments that expose tumours to high concentrations of antineoplastic drugs and other anti-tumour agents would be an advance in therapy for cancer in the liver. Moreover, it would be desirable to have a method that substantially reduces the disease progression in a patient. There is described herein a process which provides such advantages.

### SUMMARY OF THE INVENTION

Accordingly, the present invention provides a method of treating neoplasia in a subject in need of treatment, by administering to the subject an amount of a combination of 5-fluorouraoil and leucovorin effective to treat a neoplasia, in combination with SIRT, wherein a synergistic antineoplastic effect results.

Preferably, the method is used for treating a patient with colorectal liver metastases.

The present invention further provides a synergistic combination of antineoplastic agents, comprising an effective antineoplastic amount of 5-FU and LV and an amount of radionuclide-doped microparticles suitable for use in SIRT for treatment of a neoplastic growth. Preferably, the combination is prepared for use in treating a patient with colorectal liver metastases.

The invention also relates to pharmaceutical composition comprising an effective antineoplastic amount of 5-FU and LV and an amount of radionuclide-doped microparticles suitable for use in SIRT for treatment of a neoplastic growth. Preferably, the pharmaceutical composition is prepared for use in treating a patient with colorectal liver metastases.

The invention further relates to a kit for killing neoplastic cells in a subject having neoplastic calls. The kit comprises an effective antineoplastic amount of 5-FU and LV and an amount of radionuclide-doped microparticles suitable for use in SIRT for treatment of a neoplastic growth. The kit may further comprise an instructional material. Preferably, the kit is prepared for use in treating a patient with colorectal liver metastases.

The invention still further relates to use of an effective antineoplastic amount of 5-FU and LV and an amount of radionuclide-doped microparticles suitable for use in SIRT, for manufacture of a medicament for killing neoplastic cells in a subject having neoplastic cells. Preferably, the medicament is prepared for use in treating a patient with colorectal liver metastases.

The invention yet further relates to the use of an effective antineoplastic amount of 5-FU and LV and an amount of radionuclide-doped microparticles suitable for use in SIRT, for manufacture of a kit for killing neoplastic cells in a subject having neoplastic cells. Preferably, the 5-FU and LV and radionuclide-doped microparticles are manufactured for use in a kit for treating a patient with colorectal liver metastases.

Other aspects and advantages of the invention will become apparent to those skilled in the art from a review of the ensuing description.

### DETAILED DISCLOSURE OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively, and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

All references cited, including patents or patent applications are hereby incorporated by reference. No admission is made that any of the references constitute prior art.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout.

### DESCRIPTION OF PREFERRED EMBODIMENTS

Surprisingly applicants have found that the co-administration of systemic chemotherapy and SIRT to a subject, potentates the radiation from SIRT, and also has an effect on extra-hepatic disease.

Accordingly, the present invention provides a method of treating neoplasia in a subject in need of treatment, by administering to the subject an amount of a combination of 5-fluorouracil and leucovorin effective to treat a neoplasia, in combination with SIRT, wherein a synergistic antineoplastic effect results.

The present invention provides a method of treating neoplasia in a subject in need of treatment. As used herein, “neoplasia” refers to the uncontrolled and progressive multiplication of cells under conditions that would not elicit, or would cause cessation of, multiplication of normal cells. Neoplasia results in the formation of a “neoplasm”, which is defined herein to mean any new and abnormal growth, particularly a new growth of tissue, in which the growth is uncontrolled and progressive. Malignant neoplasms are distinguished from benign in that the former show a greater degree of anaplasia, or loss of differentiation and orientation of cells, and have the properties of invasion and metastasis. Thus, neoplasia includes “cancer”, which herein refer to a proliferation of cells having the unique trait of loss of normal controls, resulting in unregulated growth, lack of differentiation, local tissue invasion, and metastasis. Neoplasias for which the present invention will be particularly useful include, without limitation, colorectal liver metastases including primary and secondary liver metastases and brain metastases.

In the method of the present invention, 5-FU and LV is administered to a subject in combination SIRT, such that a synergistic antineoplastic effect is produced. A “synergistic antineoplastic effect” refers to a greater-than-addictive antineoplastic effect that is produced by a combination of chemotherapeutic drugs and SIRT, which exceeds that which would otherwise result from individual therapy associated with either therapy alone. Treatment with 5-FU and LV in combination with SIRT unexpectedly results in a synergistic antineoplastic effect by providing greater efficacy than would result from use of either of the antineoplastic agents alone.

In the method of the present invention, administration of 5-FU and LV “in combination with” SIRT refers to co-administration of the two antineoplastic treatments. Co-administration may occur concurrently, sequentially, or alternately. Concurrent co-administration refers to administration of both 5-FU and LV and SIRT at essentially the same time. For concurrent co-administration, the courses of treatment with 5-FU and LV and with SIRT may also be run simultaneously. For example, a single, combined formulation of 5-FU and LV, in physical association with SIRT, may be administered to the subject.

In the method of the present invention, 5-FU and LV therapy and SIRT also may be administered in separate, individual treatments that are spaced out over a period of time, so as to obtain the maximum efficacy of the combination. When spaced out over a period of time, administration of 5-FU and LV is preferably given to a patient for a period of time such as 1 to 10 days, but more preferably about 3 to 5 days following which SIRT is applied. This cycle may be repeated as manner times as necessary and as long as the subject is capable of receiving said treatment.

As used herein “treatment” includes:

(I) preventing a disease, disorder or condition from occurring in an subject which may be predisposed to the disease, disorder and/or condition but has not yet been diagnosed as having it;

(II) inhibiting the disease, disorder or condition, i.e., arresting its development; or

(III) relieving the disease, disorder or condition, i.e., causing regression of the disease, disorder and/or condition.

In the method of the present invention, neoplasia is treated in a subject in need of treatment by administering to the subject an amount of a combination of 5-FU and LV effective to treat a neoplasia in combination with a sufficient amount of SIRT to treat a neoplasia, wherein a synergistic antineoplasia effect results.

The subject is preferably a mammal (e.g., humans, domestic animals, and commercial animals, including cows, dogs, monkeys, mice, pigs, and rats), and is most preferably a human.

5-FU and LV Chemotherapy

In the method of the present invention, an amount of 5-FU and LV that is “effective to treat the neoplasia” is an amount that is effective to ameliorate or minimize the clinical impairment or symptoms of the neoplasia, in either a single or multiple dose of 5-FU and LV when combined with SIRT. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasm; or by limiting, suspending, terminating, or otherwise controlling the maturation and proliferation of cells in the neoplasm. Notably, the amounts of 5-FU and LV effective to treat neoplasia in a subject in need of treatment will vary depending on the type of SIRT used, as well as the particular factors of each case, including the type of neoplasm, the stage of neoplasia, the subject's weight, the severity of the subject's condition, and the method of administration. These amounts can be readily determined by the skilled artisan.

As an example only, doses of 5-FU administered intraperitoneally may be between 100 and 600 mg/M²/day, or between 200 mg/M²/day and 500 mg/M²/day. More preferably doses of 5-fluorouracil administered intraperitoneally will be between 300 and 480 mg/M²/day, or between 400 mg/M²/day and 450 mg/M²/day. An example being 425 mg/M²/day. Doses of LV administered intraperitoneally will usually be about one twentieth of the dose of 5-FU. So, for example if the dose of 5-FU is 425 mg/M²/day then the dose of LV will be about 20 mg/M²/day. A skilled artisan will recognise appropriate levels of LV.

5-FU and LV treatment according to the present invention may be administered to a subject by known procedures, including, but not limited to, oral administration, parenteral administrative (e.g., intramuscular, intraperitoneal, intravascular, intravenous, or subcutaneous administration), and transdermal administration. Preferably, the 5-FU and LV agents are administered parenterally.

For parenteral administration, the formulations of 5-FU and LV (whether individual or combined) may be combined with a sterile aqueous solution that is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile.

The formulations may be presented in unit or multi-dose containers, such as sealed ampoules or vials. Moreover, the formulations may be delivered by any mode of injection, including, without limitation, epifascial, intracapsular, intracutaneous, intramuscular, intraorbital, intraperitoneal (particularly in the case of localized regional therapies), intraspinal, intrasternal, intravascular, intravenous, parenchymatous, or subcutaneous.

SIRT Therapy

According to the invention the person skilled in the art will appreciate that SIRT may be applied by any of a range of different methods, some of which are described in U.S. Pat. Nos. 4,789,501, 6,011,677, 5,302,369, 6,296,831, 6,379,648, or WO applications 200045826, 200234298 or 200234300. Accordingly administration of radionuclide doped microparticles may be by any suitable means, but preferably by delivery to the relevant artery. For example in treating liver cancer, administration is preferably by laparotomy to expose the hepatic artery or by insertion of a catheter into the hepatic artery via the femoral, or brachial artery. Pre or co-administration of another agent may prepare the tumour for receipt of the particulate material, for example a vasoactive substance, such as angiotension-2 to redirect arterial blood flow into the tumour. Delivery of the particulate matter may be by single or multiple doses, until the desired level of radiation is reached.

The radionuclide doped microparticles need not be limited to any particular form or type of microparticle. So, for example, the radionuclide doped microparticles suitable for use in the invention may comprise any material capable of receiving a radionuclide such as through impregnation, absorbing, coating or more generally bonding the particles together.

In one particular form of the invention the microparticles are prepared as polymeric particles. In another form of the invention the microparticles are prepared as ceramic particles (including glass).

Where the microparticles are prepared as polymeric matrix they will preferably have a stably incorporated radionuclide. More preferably the radionuclide will be incorporated by precipitation of the radionuclide as a salt. A description of such particles including methods for there production and formulation as well as there use is provided in co-owned European application number 200234300, of which the teachings therein are expressly incorporated herein by reference.

Where the microparticles are ceramic particles (including glass) the selected particles will usually possess the following properties:

(1) the particles will generally be biocompatible, such as calcium phosphate-based biomedical ceramics or glass.

(2) the particles will generally comprise a radionuclide that preferably has sufficiently high energy and an appropriate penetration distance, which are capable of releasing their entire energy complement within the tumour tissue to effectively kill the cancer cells and to minimize damage to adjacent normal cells or to attending medical personnel. The level of radiation activity of the ceramic or glass will be selected and fixed based upon the need for therapy given the particular cancer involved and its level of advancement. The ideal half-life of the radionuclides is somewhere between days and months. On the one hand, it is impractical to treat tumours with radionuclides having too short a half-life, this characteristic limiting therapy efficiency. On the other hand, in radiotherapy it is generally difficult to trace and control radionuclides having a long half-life.

(3) Third, the particles must be of a suitable size. The size of the particles for treatment depends upon such variables as the surface area of the tumour, capillary permeability, and the selected method of introduction into the tumour (i.v. versus implant by surgical operation).

(4) Fourth, some ceramic processes involve inclusion of extraneous substances as contaminants that might produce undesired radionuclides. Should these be well taken care of, the size of the particles can then be controlled by granulation and meshing.

There are many processes for producing small granular ceramic or glass particles. One of these involves the introduction of small amounts of the ceramic particles passing through a high-temperature melting region. Ceramic spherules are yielded by surface tension during melting. After the solidification, condensation, collection and sorting processes, ceramic spherules of various sizes can be obtained. The particle size of ceramic spheroid can be controlled by the mass of granules introduced into the high-temperature melting region or can be controlled by collecting spheroids of various sizes through the selection of sedimentary time during liquid-sedimentation.

The ceramic or glass materials for preparing those particles can be obtained commercially or from ultra-pure ceramic raw materials if the commercial products do not meet specifications for one reason or another. The ceramic or glass particles for radiation exposure in this invention can be yielded by traditional ceramic processes, which are well known by those skilled in this art. The ceramic processes such as solid-state reaction, chemical co-precipitation, sol-gel, hydrothermal synthesis, glass melting, granulation, and spray pyrolysis can be applied in this invention for the production of specific particles.

The ceramic or glass particles of suitable size which are obtained commercially or which are produced by the processes described above are washed twice with distilled water. Then the supernate is decanted after sedimentation for 3 minutes. The above two steps are repeated 3 times to remove the micro-granules adhering on the surfaces of the particles. Then a certain amount of ceramic or glass particles prepared from the processes described above are introduced into a quartz tube. After being sealed, the quartz tube is placed inside a plastic irradiation tube, then the irradiation tube is closed. The irradiation tube is put into a vertical tube of the nuclear reactor and the multiple tube assembly is irradiated with an approximated neutron flux for an approximated exposed period (e.g., for about 24 to about 30 hours). Following exposure, the irradiation tube is taken out of the nuclear reactor for cooling. According to this method, ceramic or glass particles carrying radionuclides can be generated.

The microparticles of the invention be they polymer or ceramic based can be separated by filtration or other means known in the art to obtain a population of microparticles of a particular size range that is preferred for a particular use. The size and shape of the microparticles is a factor in the distribution and drug delivery in the tissues.

When small particles are administered into the arterial blood supply of a target organ, it is desirable to have them of a size, shape and density that results in the optimal homogeneous distribution within the target organ. If the small particles do not distribute evenly then they may accumulate in excessive numbers in some areas and cause local areas of excessive radiation. The particulate material is preferably low density, more particularly a density below 3.0 g/cc, even more preferably below 2.8 g/cc, 2.5 g/cc, 2.3 g/cc, 2.2 g/cc or 2.0 g/cc. The ideal particle for injection into the blood stream would have a very narrow size range with a standard deviation of less than 5%, so as to assist in even distribution of the microparticles within the target organ, particularly within the liver and would be sized in the range 5-200 micron preferably 15-100 micron and preferably 17-50 micron, more preferably 20-35 microns and most preferably 25 to 35 microns.

The radionuclide which is incorporated into the microparticles in accordance with the present invention is preferably yttrium-90, but may also be any other suitable radionuclide such as holmium, samarium, iodine, phosphorous, indium and rhenium.

The amount of microparticles used in the method and which will be required to provide effective treatment of a neoplastic growth will depend substantially on the radionuclide used in the preparation of the microparticles. By way of example, an amount of yttrium-90 activity that will result in an inferred radiation dose to the normal liver of approximately 80 Gy may be delivered. Because the radiation from SIRT is delivered as a series of discrete point sources, the dose of 80 Gy is an average dose with many normal liver parenchymal cells receiving much less than that dose. Alternate doses of radiation may be delivered depending on the disease state and the physician's treatment needs. Such variation of radiation doses by altering the amount of microparticles used will be something that a skilled artisan will know how to determine.

The term microparticle is used in this specification as an example of a particulate material, it is not intended to limit the invention to microparticles of any particular shape or configuration. A person skilled in the art will however appreciate that the shape of the particulate material while preferably be without sharp edges or points that could damage the patients arteries or catch in unintended locations.

Preferably the particulate material is substantially spherical, but need not be regular or symmetrical in shape.

It is also desirable to have the particulate material manufactured so that the suspending solution has a pH less than 9. If the pH is greater than 9 then this may result in irritation of the blood vessels when the suspension is injected into the artery or target organ. Preferably the pH is less than 8.5 or 8.0 and more preferably less than 7.5.

In a highly preferred form or the invention there is provided a method for treating neoplasia in a subject in need of treatment, by administering an effective antineoplastic amount of 5-FU and LV as described above in combination with an amount of radionuclide-doped microparticles suitable for use in SIRT for treatment of a neoplastic growth, wherein a synergistic antineoplasia effect results.

In addition to the identified chemotherapeutic agents and radionuclide doped microparticles the invention may also include an effect treatment of immunomodulators as part of the therapy. Illustrative immunomodulators suitable for use in the invention are alpha interferon, beta interferon, gamma interferon, interleukin-2, interleukin-3, tumour necrosis factor, granulocyte-macrophage colony stimulating factor, and the like.

The present invention further provides a synergistic combination of antineoplastic agents, comprising an effective antineoplastic amount of 5-FU and LV and an amount of radionuclide-doped microparticles suitable for use in SIRT for treatment of a neoplastic growth. Preferably, the combination is prepared for use in treating a patient with colorectal liver metastases.

The invention also relates to pharmaceutical composition comprising an effective antineoplastic amount of 5-FU and LV and an amount of radionuclide-doped microparticles suitable for use in SIRT for treatment of a neoplastic growth. Preferably, the pharmaceutical composition is prepared for use in treating a patient with colorectal liver metastases.

The invention further relates to a kit for killing neoplastic cells, in a subject having neoplastic cells. The kit comprises an effective antineoplastic amount of 5-FU and LV as described above and an amount of radionuclide-doped microparticles as described above suitable for use in SIRT for treatment of a neoplastic growth.

The kit may further comprise an instructional material. Preferably, the kit is prepared for use in treating a patient with colorectal liver metastases.

The invention still further relates to use of an effective antineoplastic amount of 5-FU and LV as described above and an amount of radionuclide-doped microparticles as described above suitable for use in SIRT, for manufacture of a medicament for killing neoplastic cells in a subject having neoplasm cells. Preferably, the medicament is prepared for use in treating a patient with colorectal liver metastases.

The invention yet further relates to the use of an effective antineoplastic amount of 5-FU and LV as described above and an amount of radionuclide-doped microparticles as described above suitable for use in SIRT, for manufacture of a kit for killing neoplastic cells in a subject having neoplastic cells. Preferably, the 5-FU and LV and radionuclide-doped microparticles are manufactured for use in a kit for treating a patient with colorectal liver metastases.

### BEST MODE(S) FOR CARRYING OUT THE INVENTION

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

Patients: Patients with colorectal liver metastases either with, or without, extra-hepatic metastases were eligible for trial entry. Patients were required to be greater than 18 years of age, have histologically proven adenocarcinoma of the colorectum, unequivocal CT scan evidence of liver metastases that could not be treated by resection or any locally ablative technique, not have received chemotherapy or radiotherapy for the liver metastases, have adequate hematologic, hepatic and renal function, no CNS metastases and no evidence of cirrhosis, ascites or portal hypertension and a WHO performance status <3. Patients were randomised to receive systemic chemotherapy either alone (control arm), or with the addition of a single administration of SIR-Spheres® (SIRTeX Medical Ltd). All patients had multiple bi-lobar liver metastases and were reviewed in a surgical oncology unit to confirm that the metastases were so advanced that they were unable to be treated by any form of local ablation.

The primary aims of this study was to compare the response rate and toxicity from adding a single treatment with SIR-Spheres® to a standard regimen of fluorouracil fleucovorin chemotherapy. Patients were entered into the study from three Australian hospitals and were stratified prior to randomisation by institution, presence or absence of extra-hepatic metastases and extent of liver involvement (> or < than 25%) by tumour. In order to stratify for extent of liver involvement, the tumour and liver volumes were calculated form the seal slices of the pre-randomisation CT scan and recorded as a tumour/total liver volume ratio as described previously (Ettinger D, et al (1985) Am J Clin Oncol 8, 413-418). All patients were fully informed of the nature of the trial and signed informed consent to enter the study. The trial was approved by the Human Ethics Committees of participating institutions and conformed to the Australian National Health & Medical Research Council Statement on Human Experimentation and the World Medical Association Declaration of Helsinki.

Investigations: All patients underwent a pre-treatment spiral CT scan of the whole abdomen and either a CT scan of the chest or chest X-ray and blood tests to assess haematologic, renal and liver function and serum CEA.

Patients randomised to treatment with SIRT underwent a trans-femoral hepatic anglogram to assess the arterial anatomy of the liver and to plan the subsequent administration of SIR-Spheres®. Patients randomised to treatment with SIRT also underwent a nuclear medicine planar and SPECT scans to estimate the amount of SIR-Spheres® that would pass through the liver and lodge in the lungs. This was performed by injecting technetium-99 labelled macro-aggregated albumin (MAA) into the hepatic artery at the time of the angiogram and measuring the radioactivity in the liver and lungs using a gamma camera. Areas of interest were drawn around the liver and lungs and the percentage of the MAA that lodged in the lungs was determined as a fraction of the total amount of MAA in both lungs and liver. This was recorded as a ‘percentage lung break-through’ in order to decide whether to reduce the amount of yttrium-90 activity to administer to the patient. Previous experiments had shown that a lung break-through percentage of >13% might result in radiation pneumonitis and should be accompanied by a reduction in the amount of yttrium-90 activity administered to the patient (Ho S et al (1996) Europ J Nuclear Med. 23, 947-952). This technique has been shown to be a reliable method for determining the subsequent distribution of SIR-Spheres®. Patients were followed after trial entry with three monthly clinical evaluation and quality of life assessment (QoL), three-monthly CT scans of the abdomen and either a plain X-ray or CT scan of the chest and monthly serologic tests of haematologic, liver and renal function and CEA. Patients found to have obtained a complete (CR) or partial (PR) response on CT scan had a second confirmatory CT scan at not less than 4 weeks after the initial scan that showed the response.

Randomisation, Data Handling and Recording of Response and Toxicity: Patient registration and randomisation was made by telephoning the independent Australian National Health & Medical Research Council Clinical Trials Centre which randomised patients using a computer based program. All source data for this trial has been monitored and audited before being subjected to analysis and interpretation. All serial CT scans were read by an independent person not associated with the trial, who was blinded to the treatment group of patients and who is experienced in reporting CT scans of the liver.

Response was determined using RECIST criteria (Therasse P et al (2000) J Natl Cancer Inst 92, 205-216). The RECIST criteria were developed with particular application for reporting the results of phase 2 trials and result in very similar response outcomes as the conventional WHO method.

Toxicity was recorded on all patients using standard UICC recommendations for grading of acute and subacute toxicity criteria.

Quality of life was measured at randomisation and then 3 monthly using the validated 23 point FLIC questionnaire (Therasse P, et al (2000) J Natl Cancer Inst 92, 205-216). In addition, clinicians completed an assessment of the patients' well being at the same intervals using the Spitzer index (Spitzer W O, et a (1981) Journal of Chronic Diseases. 34(12). 585-97).

Protocol Treatment: Patients randomised to both arms were treated with 5-fluorouracil 425 mg/M²/day plus leucovorin 20 mg/M²/day for 5 consecutive days and repeated at 4 weekly intervals. Chemotherapy cycles were continued in both patient groups until evidence of unacceptable toxicity, patient request or disease progression.

Patients in the experimental arm received a single dose of SIR-Spheres® that was administered on the 3^{rd} or 4^{th} day of the second cycle of chemotherapy. The SIR-Spheres® was administered into the hepatic artery via a trans-femoral catheter that was placed under local anaesthetic. In patients where there was more than one hepatic artery supplying blood to the liver, the catheter was repositioned during administration and the total dose of SIR-Spheres® was divided into separate aliquots depending on the estimated volume of tumour being supplied by each feeding artery. Patients treated with SIRT were generally kept in hospital overnight and discharged home the following day. As Angiotensin-2 has been shown to increase the microparticle targeting of tumours within the liver, a single bolus of 25 ug of Angiotensin-2 was pulsed into the hepatic artery 30 seconds before administering the SIR-Spheres®.

The first five patients treated with SIRT received a standard dose of 25 GBq of yttrium-90 activity. As one very small patient developed evidence of radiation hepatitis at this radiation dose, the subsequent six patients were treated with a dose of SIR-Spheres® that was calculated from the patient's body surface area and the size of the tumour within the liver according to the following equation:

$$Dose of SIR \text{-} {Spheres}^{®} in GBq = ( {BSA}^{*} - 0.2 ) + ( \frac{\% tumour involvement}{100} )$$

* BSA=body surface area measured in square metres

Non-Protocol Treatment: Once protocol treatment ceased, further cancer specific treatment, including non-protocol chemotherapy, was allowed to best manage patient care. All non-protocol cancer specific treatment was recorded in all patients. Other supportive, but not cancer specific treatment was allowed for patient management.

Statistical Analysis: The trial was designed to enter 18 patients and closed after entering 21 patients. Outcome criteria were analysed on an intention-to-treat basis end all tests are two-tailed. Patients who died without having follow-up scans or in whom progressive disease (PD) was not logged on CT scans were recorded as having PD at the time of death. The chemotherapy dose intensity is defined as the average amount of chemotherapy of each cycle expressed as a percentage of the amount given in the first cycle. Time to disease progression curves were constructed using the method of Kaplan-Meier and compared using the logrank test (Tibshirani, R. (1982) Clinical and Investigative Medicine. 5, 63-68). Response comparisons were performed using the Kruskal Wallis test whilst the t-test was used to compare difference in the quality of life measures.

### RESULTS

Patients: Ten patients were randomised to receive chemotherapy alone and eleven to receive the combination treatment. One patient in the control am continued to have stable disease (SD) and all other patients in both groups have logged either a Best Confirmed Response to treatment, PD or have died. No patients in the chemotherapy arm and five patients in the combination arm continue to receive protocol treatment. Three patients in the chemotherapy arm had extrahepatic metastases (two in lung, one in peritoneal cavity) and two in the combination arm (both in lung). Table 1 details the patient and tumour profiles of patients. There is no significant difference in any of the tumour or patient characteristics between the two groups.

TABLE 1
Patient and Tumor Characteristics

| | Chemotherapy | SIRT + Chemotherapy |
|---|---|---|
| No of patients | 10 | 11 |
| Mean Age (years) | 65 | 64 |
| Male/Female | 6/2 | 10/1 |
| Extrahepatic metastases | 3 | 2 |
| Histologic differentiation of | 2/6/2 | 1/10/0 |
| primary | | |
| bowel cancer: | | |
| poor/moderate/well | | |
| Size of Liver metastases | | |
| <25% | 7 | 8 |
| >25% | 3 | 3 |

Protocol Treatment: Two patients in the chemotherapy arm refused treatment, deteriorated rapidly and died at 30 days and 45 days after trial entry. The remaining 8 patients were treated with protocol chemotherapy, of whom one continues on treatment, one has refused further treatment and six had treatment stopped due to disease progression.

Of the eleven patients in the combination arm, all received at least one cycle of chemotherapy and treatment with SIRT. Five patients continued to receive protocol chemotherapy and have long-term responses, four had it stopped due to disease progression and two patients died without evidence of disease progression.

The number of cycles of protocol chemotherapy was greater for patients receiving the combined treatment. Apart from the two patients that were early deaths in the control arm and did not receive any chemotherapy, the reason for this difference was because most patients in the combined treatment arm experienced a prolonged response and therefore continued to receive ongoing treatment. However, the dose intensity was slightly higher for patients treated with chemotherapy alone, indicating that the lower response rate for patients in the control arm was not due to less intensive chemotherapy.

For patients treated with SIRT plus chemotherapy, the initial five were treated with standard amount of 2.5 GBq and the following six patients had their SIR-Spheres® dose individualised. These six patients received from 1.5 GBq to 2.1 GBq of yttrium-90 activity.

TABLE 2
Protocol Treatment Administered (as at 10^{th} Sept 01)

| | | SIRT + |
|---|---|---|
| | Chemotherapy | Chemotherapy |
| Total number of chemotherapy cycles | 38 | 89 |
| Fluorouracil Dose Intensity (%) | 92.0 | 85.4 |
| Mean SIR-Spheres ® dose | NA | 2.25 GBq |

Response: Using the RECIST criteria, the First Integrated Response and Best Combined Response are shown in Tables 2A and 2B. Although several patients in the chemotherapy arm showed minor diminution in tumour size with treatment, no patient qualified for a response. No complete responses were recorded in either group. However, in several patients treated with SIRT plus chemotherapy, all CT evidence of tumour disappeared and was replaced with small dense calcifications on serial CT scans. As there was no way of knowing if any viable tumour remained in these calcified deposits, these patients were determined to have partial, rather than complete, responses.

Ten of 11 patients treated with SIRT plus chemotherapy showed a partial response on at least one CT scan and in 8 of these patients it was confirmed by a second follow-up CT scan. Two patients that registered an initial PR did not get a confirmatory CT scan. One patient had a profound reduction in tumour size on the first follow-up CT scan but died from sepsis associated with chemotherapy induced neutropenia. The second patient also registered a PR but declined further follow-up and did not get a confirmatory CT scan. These two patients were logged as SD for the Best Confirmed Response. There was no difference in response rate between patients receiving either a standard 2.5 GBq of yttrium-90 activity or when the dose was individualised.

TABLE 2A
Response Data. First INTEGRATED RESPONSE

| | CR | PR | SD | PD |
|---|---|---|---|---|
| Chemotherapy n = 10 | 0 | 0 | 6 | 4 |
| SIRT + Chemotherapy n = 11 | 0 | 10 | 1 | 0 |

Comparison between groups p<0.001

TABLE 2B
Response Data. BEST CONFIRMED RESPONSE

| | CR | PR | SD | PD |
|---|---|---|---|---|
| Chemotherapy n = 10 | 0 | 0 | 6 | 4 |
| SIRT + Chemotherapy n = 11 | 0 | 8 | 3 | 0 |

Comparison between groups p<0.001

Time to Progressive Disease: Progressive disease (PD) is a measure that treatment is no longer effective. At the time of this report, 8 of 10 patients in the control arm and 4 of 11 patients in the experimental arm have recorded PD. The time to PD was significantly longer for patients treated with the combination of SIRT plus chemotherapy as shown in Table 3.

TABLE 3
Time to Progressive Disease (as at 10^{th} Sept 01)

| | Median (months) |
|---|---|
| Chemotherapy | 3.4 |
| SIRT + Chemotherapy | 15.6 |

Comparison between groups p<0.0005

Site of Progressive Disease: The site of first disease progression was recorded for all patients. In the chemotherapy arm the site of first progression was the liver in six patients, bone in one patient and two patients died before PD was recorded on any imaging study. In the SIRT plus chemotherapy arm the site of first disease progression was the liver in three patients, liver and lung in one patient and two patients died before PD was recorded on any imaging study.

Toxicity and Adverse Events: There was one treatment related death in the combined treatment group. This patient received four cycles of chemotherapy and experienced chemotherapy induced neutropenia with each cycle despite progressive chemotherapy dose reductions. On the fourth occasion, he rapidly deteriorated and died from sepsis associated with the neutropenia. One patient treated with SIRT plus chemotherapy developed a liver abscess in the site of a necrotic tumour mass following treatment. The patient recovered quickly after drainage of the abscess. One patient developed radiation induced liver Cirrhosis at approximately one year after start of treatment. This patient had a long term response to treatment and the signs and symptoms associated with the cirrhosis improved with conservative treatment. This patient remains alive and without symptoms at 26 months from randomisation. As this patient weighed 43 kg, treatment with 2.5 GBq of yttrium-90 activity was considered excessive. As a result of this experience, future patients were treated with an amount of SIR-Spheres® that was calculated from the size of the patient and tumour. Four patients developed transient abdominal pain at the time of injection of the SIR-Spheres® that resolved with narcotic analgesia. Treatment related toxicity is shown in Table 4.

TABLE 4
Grade 3 & 4 Toxicity Experienced during Protocol Treatment

| | | SIRT + |
|---|---|---|
| | Chemotherapy | Chemotherapy |
| Number of Grade 3-4 toxicity events | | |
| granulocytopenia | 0 | 3 |
| nausea, vomiting | 1 | 1 |
| mucositis | 1 | 4 |
| gastritis | 1 | 1 |
| diarrhoea | 1 | 2 |
| anorexia | 1 | 0 |
| cirrhosis | 0 | 1 |
| liver abscess | 0 | 1 |
| Total number of events | 6 | 13 |

Quality of Life: Changes from baseline patient rated quality of life for the first three months of treatment was analysed using the t-test. The number of quality of life assessments in the chemotherapy only arm diminished after three months due to disease progression. Changes in the quality of life were almost identical in both arms (p 0.96). This was also the case for physician rated quality of life (p=0.98). This lack of variation was mainly due to the fact that most patients were still receiving chemotherapy during this three-month period.

### DISCUSSION

Selective Internal Radiation Therapy (SIRT) is a relatively new technique for the treatment of advanced primary and secondary liver cancer. The technique involves injecting radioactive SIR-Spheres® into the arterial supply of the liver, following which the microparticles concentrate in the micro-vasculature of the tumour, as opposed to the normal liver parenchyma. As the microparticles contain the high energy beta emitting radionuclide yttrium-90, this results in the tumour being irradiated to high doses, while the radiation delivered to normal liver tissue is maintained at a tolerable level.

Because both primary and metastatic liver tumours are supplied almost entirely by blood from the hepatic artery, as opposed to the normal liver parenchyma, the SIR-Spheres® concentrate preferentially in the tumour compartment within the liver (Archer S (1989) Brit J. Surgery 76, 545-548). This physiological tumour targeting can be enhanced by injecting Angiotensin-2 into the hepatic artery immediately before the administration of the SIR-Spheres. For a period of several minutes the Angiotensin-2 causes the arteries of the normal liver to constrict but not those supplying the tumour (Burton M A, (1988) Europ. J. Cancer Clin. Oncol. 24(8), 1373-1376; Burton M, el al (1985) Cancer Research. 45, 5390-5393), If the SIR-Spheres are injected during this time, they will further concentrate within the tumour microvasculature.

Initial clinical studies have shown that treatment of liver metastases from primary colorectal cancer with SIR-Spheres® alone results in a high rate of tumour regression (13). In later trials SIRT was combined with ongoing cycles of hepatic artery chemotherapy with improved effect (Gray B. et al (1992) Aust NZ J Surgery. 62, 105-110; Gray B N, et al Gl Cancer. 3(4). 249-257).

The findings from this study show that the addition of a single administration of SIR-Spheres® to a regimen of FU/LV chemotherapy greatly increases both the response rate and length of that response. Although no patient in the control arm obtained a response, many had stabilisation of their disease. The high response rate and long time to disease progression in the combined treatment arm are far higher than reported with other treatment regimens and is also greater than that reported for patients treated with SIRT plus HAC. There was more grade 3-4 toxicity in patients receiving the combination treated and this is largely due to the greater period that these patients received protocol treatment. The one treatment related death in this study was due to the chemotherapy rather than the SIRT. The quality of life of patients is also not compromised in the short term by the addition of SIRT.

1. A method of treating neoplasia in a subject in need of treatment, by administering to the subject an amount of a combination of 5-fluorouracil and leucovorin effective to treat a neoplasia, in combination with SIRT, wherein a synergistic antineoplastic effect results.

2. The method according to claim 1 wherein the neoplasia treated is a colorectal liver metastases.

3. A synergistic combination of antineoplastic agents, comprising an effective antineoplastic amount of 5-FU and LV and an amount of radionuclide-doped microparticles suitable for use in SIRT for treatment of a neoplastic growth.

4. A composition according to claim 3 wherein the neoplastic growth is a colorectal liver metastases.

5. A pharmaceutical composition comprising an effective antineoplastic amount of 5-FU and LV and an amount of radionuclide-doped microparticles suitable for use in SIRT for treatment of a neoplastic growth.

6. A pharmaceutical composition according to claim 5 wherein the composition is formulated for the treatment of a colorectal liver metastases.

7. A kit for killing neoplastic cells in a subject having neoplastic cells, the kit comprising an effective antineoplastic amount of 5-FU and LV and an amount of radionuclide-doped microparticles suitable for use in SIRT for treatment of a neoplastic growth.

8. A kit according to claim 7 that further comprises instructional material.

9. A kit according to claim 7 wherein the amount of 5-FU and LV is formulated for the treatment of colorectal liver metastases.

10. Use of an effective antineoplastic amount of 5-FU and LV and an amount of radionuclide-doped microparticles suitable for use in SIRT, for the manufacture of a medicament for killing neoplastic cells in a subject having neoplastic cells.

11. Use of an effective antineoplastic amount of 5-FU and LV and an amount of radionuclide-doped microparticles suitable for use in SIRT, for the manufacture of a kit for killing neoplastic cells in a subject having neoplastic cells.
